(19) United States
(12) Patent Application Publication
(10) Pub. No.: US 2024/0398820 A1
(43) Pub. Date: Dec. 5, 2024

(54) METHODS FOR TREATMENT OF PULMONARY HYPERTENSION

(71) Applicant: Respira Therapeutics, Inc., Palo Alto, CA (US)

(72) Inventor: Jeffry Weers, Half Moon Bay, CA (US)

(73) Assignee: Respira Therapeutics, Inc., Palo Alto, CA (US)

(21) Appl. No.: 18/679,971

(22) Filed: May 31, 2024

Related U.S. Application Data
(60) Provisional application No. 63/470,091, filed on May 31, 2023.

(51) Int. Cl. A61K 31/53; A61K 9/00

(52) U.S. Cl. CPC A61K 31/53; A61K 9/007

(57) ABSTRACT

According to embodiments of the invention, a method of treating pulmonary hypertension comprises administering to a subject in need thereof an effective amount of vardenafil or a pharmaceutically acceptable salt or hydrate thereof. According to the method, the vardenafil is administered via inhalation pro re nata using a portable inhaler, and the vasodilator is administered at least 2-30 minutes before physical exertion.

### CROSS-REFERENCE TO RELATED APPLICATION

This application claims the benefit of provisional U.S. patent application No. 63/470,091, filed 31 May 2023 and titled “Methods for Treatment of Pulmonary Hypertension”, which is incorporated herein by reference in its entirety.

### FIELD OF THE INVENTION

This invention relates to methods and compositions useful in subjects with pulmonary arterial hypertension and to devices useful in connection with such methods and compositions.

### BACKGROUND

Pulmonary hypertension (PH) is a rare disease defined by abnormally high pulmonary arterial pressure and pulmonary vascular resistance (PVR). Group 1 PH, pulmonary arterial hypertension (PAH), is a progressive disease of unknown etiology. It may be characterized by a mean pulmonary artery pressure (mPAP)≥20 mmHg, pulmonary capillary wedge pressure (PCWP)≤15 mmHg, and pulmonary vascular resistance (PVR) of ≥3 Wood units, as well as physiological changes to the pulmonary arteries. As the disease progresses, exercise capacity declines and daily activities become more difficult.

There is currently no cure for PAH. The disease is managed by approved treatments, such as endothelin receptor agonists, phosphodiesterase 5 inhibitors (PDE5i), and prostacyclin analogs that reduce symptoms and slow disease progression over time.

Managing the acute symptoms induced by daily activities is an ongoing challenge for patients living with PAH because none of the approved treatments can be used for acute symptom relief. Current approved therapies for PAH are chronic treatments that require multiple doses to alleviate symptoms and delay disease progression. Moreover, despite ongoing advances in these therapies, patients continue to experience a significant reduction in cardiorespiratory fitness and exercise capacity. These therapies are also associated with adverse effects that reduce patients' quality of life, including nausea, headache, and flushing, as well as injection site pain and infection with infusion medications. These adverse effects are due, in part, to the doses required for an effective local drug concentration in the pulmonary arteries.

There is therefore an urgent unmet clinical need for an as-needed (PRN) treatment for PAH that will reduce acute symptoms and allow patients to perform daily activities and exercise.

### BRIEF SUMMARY OF THE INVENTION

According to embodiments of the invention, a method of treating pulmonary hypertension comprises administering to a subject in need thereof an effective amount of vardenafil or a pharmaceutically acceptable salt or hydrate thereof. According to the method, the vardenafil is administered via inhalation pro re nata using a portable inhaler, and the vasodilator is administered at least 2-30 minutes before physical exertion.

In embodiments, administering the vardenafil comprises targeting the vardenafil to the small airways of the lungs in the subject. In further embodiments, a nominal dose of about 0.5 mg to about 1 mg of vardenafil is delivered to the subject upon inhalation. In an embodiment of the invention, a nominal dose of about 0.5 mg of vardenafil is delivered to the subject upon inhalation. Alternatively, in an embodiment of the invention, a nominal dose of about 1.0 mg of vardenafil is delivered to the subject upon inhalation.

### DETAILED DESCRIPTION OF PREFERRED EMBODIMENTS

This invention relates to methods and compositions useful in subjects with PAH and to devices useful in connection with such methods and compositions. The methods include administration of phosphodiesterase type 5 inhibitors (PDE5i) by inhalation PRN. In some embodiments, the PDE5i is vardenafil.

Methods disclosed herein provide lung-targeted therapies for patients with PAH, and other forms of PH, or other lung disorders, that are delivered to the pulmonary vascular bed, with minimal systemic exposure, via a dry powder inhaler. Such an appropriate inhaled medication would be characterized by good pulmonary tolerance and little or no systemic exposure and would lend itself to both targeted chronic maintenance therapy delivered 1, 2, 3, or more times per day and the potential for PRN use.

A PRN dosed medication for PH, if convenient to use, and portable, easily allows a patient to enhance their short-term function and exercise tolerance to accomplish day to day activities of daily living (ADL) or when wanting to perform other more vigorous activities. By avoiding the potential for ventilation/perfusion mismatching associated with systemic (enteral or parenteral) administration of pulmonary vasodilator drugs as well as the dose limiting systemic side effects, the provided methods of an inhaled pulmonary vasodilator delivered by a simple to use portable inhaler offers improved function and quality of life for patients with effort limitation due to PH and/or interstitial lung disease (ILD).

In some instances, self-administration of a lower dose of drug via inhalation, e.g., an inhaled PDE5i such as vardenafil, as a PRN therapy on top of chronic background therapies may provide patients with improved exercise tolerance throughout the day, while minimizing the risk of systemic side effects. PRN administration may suggest a drug product that is effectively targeted to the lungs and pulmonary blood vessels while minimizing the drug concentration in the systemic circulation that contributes to drops in systemic blood pressure. Aerosol administration also provides a rapid onset of action (comparable to an injection) as compared to oral administration.

PRN administration via inhalation of vasodilators offers PH patients an opportunity to optimize their daily life function by self-administration of a lower nominal dose of drug, preparatory to increased activity to augment background therapy efficacy, would offer safe and effective improvement in quality of life indices and measures of daily function.

Methods and compositions for treatment of pulmonary hypertension and other lung disorders, including methods and compositions involving, e.g., PDE5 inhibitors such as vardenafil, are disclosed in U.S. Pat. No. 10,912,778, titled “Methods for Treatment of Pulmonary Hypertension”, which is incorporated herein by reference in its entirety.

### PDE5i

PDE5 inhibitors inhibit phosphodiesterase type 5 (PDE5) enzyme, which is responsible for the degradation of cyclic guanosine monophosphate (cGMP). Pulmonary arterial hypertension is associated with impaired release of nitric oxide (NO) by the vascular endothelium and consequent reduction of cGMP concentrations in the pulmonary vascular smooth muscle. PDE5 is the predominant phosphodiesterase in the pulmonary vasculature. Inhibition of PDE5 by PDE5 inhibitors increases the concentrations of cGMP, resulting in relaxation of pulmonary vascular smooth muscle cells and vasodilation of the pulmonary vascular bed. The PDE5i class of drugs appropriate for use in connection with embodiments of the invention includes sildenafil, tadalafil, vardenafil, avanafil, benzamidenafil, lodenafil, mirodenafil, udenafil, zaprinast, and others.

In connection with one embodiment of the inventions, the PDE5i drug is vardenafil (i.e., 1-[[3-(1,4-dihydro-5-methyl-4-oxo-7-propylimidazo[5,1-f][1,2,4]triazin-2-yl)-4-ethoxyphenyl]sulfonyl]-4-ethyl-piperazine, or a pharmaceutically acceptable salt thereof, such as a monohydrochloride salt). Vardenafil and other 2-phenyl substituted imidazotriazinones are described, for example, in U.S. Pat. Nos. 6,890,922; 7,122,540; 7,314,871; 7,704,999; and 7,696,206, which are incorporated herein by reference in their entirety. Vardenafil has been shown to be safe and effective in human patients with PAH at a dose of 5 mg, administered orally twice per day. Vardenafil has been shown to have characteristics superior to sildenafil and tadalafil for use as an inhaled agent and has been formulated into a dry powder and delivered by a dry powder inhaler device as described in International PCT Appl. No. WO/2015/089105 and U.S. Patent Appl. No. 2016/0317542, which are incorporated by reference herein in their entireties. Vardenafil is a high-affinity inhibitor of PDE5 (half-maximal inhibitory concentration [IC₅₀] 0.091±0.031).

### Methods of Use of the PDE5i

The therapeutic targets for the vasodilators disclosed herein are the smooth muscle cells within the pulmonary arteries and arterioles. One goal for a PRN therapeutic may be to maximize drug delivered to the pulmonary arteries while minimizing off-target delivery of the drug to the mouth and throat where it is swallowed, to minimize concerns about safety and tolerability. For inhaled therapeutics, improvements in lung targeting may be achieved not only by more effective drug delivery to the lungs, but also by rational design and/or selection of the drug to be delivered.

The goals for a PRN therapeutic focused on pulmonary targeting may be diametrically opposed to those for an oral therapeutic. For an orally administered therapeutic the goal is to maximize and maintain concentrations of drug in the systemic circulation. This is achieved by maximizing oral bioavailability, and then maintaining systemic concentrations of ‘free’ drug by minimizing protein binding and systemic clearance. In contrast, the pulmonary PRN therapeutic seeks to maximize the residence time in the lungs, either by having prolonged binding times to receptors, by slowing dissolution of drug in epithelial lining fluid, or by developing controlled release dosage forms (e.g., liposomes). Once absorbed into the systemic circulation, the goal is to minimize systemic effects, by having the drug cleared as rapidly as possible and/or be bound to plasma proteins. Moreover, minimizing oral bioavailability is also important to minimize systemic levels of drug arising from drug that is deposited in the upper respiratory tract following inhalation. Vardenafil has a slow dissociation rate and rapid clearance, making it ideally suited to PRN use.

### Devices for Inhalation of PDE5i Powder

Pulmonary administration provides non-invasive, targeted delivery of vasodilators directly to the site of action in the lungs, thereby enhancing pulmonary selectivity and reducing adverse events related to off-target delivery. Portable aerosol delivery systems are particularly advantageous for PRN administration of vasodilators. For the purposes of this disclosure, ‘portable’ inhaler refers to an inhaler that easily fits in a pocket or purse. A portable inhaler with a short administration time may be used discreetly in a public place. Methods and compositions according to embodiments of the invention may employ drug delivery devices that are portable, simple, and convenient to use (e.g., having no power source requirements, no active agent reconstitution steps, and no cleaning requirements), allowing for short administration times and a low daily treatment burden.

Administration of the compositions disclosed herein can be carried out with various classes of portable inhalers, including dry powder inhalers, pressurized metered dose inhalers, and smart-mist inhalers.

In some instances, a high efficiency dry powder inhaler (DPI) may be used to deliver the vasodilator to the patient. In some instances, the inhaler is one as described in U.S. Pat. Nos. 8,651,104; 8,561,609; U.S. Patent Appl. No. 2013/0213397, U.S. Patent Appl. No. 2015/0246189, U.S. Patent Appl. No. 2013/0340747; and U.S. Patent Appl. No. 2015/0314086, each of which are incorporated herein by reference in their entireties for all purposes. Such inhalers may enhance the delivery of dry powder compositions of many drugs and, in some cases, pure micronized drugs, including, for example, vardenafil, a PDE5 inhibitor, or, for example, powdered vardenafil hydrochloride.

In certain aspects, methods for aerosolizing dry powder compositions are provided. As a first step, a carrier-based powder pharmaceutical composition comprising a vasodilator (e.g., PDE5 inhibitor, or a pharmaceutically acceptable salt, hydrate, or ester thereof) is provided. In a second step, an inhaler may be provided, the inhaler comprising a dispersion chamber having an inlet and an outlet, and the dispersion chamber containing an actuator that is movable and reciprocatable along a longitudinal axis of the dispersion chamber. The first and second steps may be performed in any order or simultaneously. In a third step, air flow is induced through the outlet channel to cause air and the powder pharmaceutical composition to enter the dispersion chamber from the inlet, and to cause the actuator to oscillate within the dispersion chamber to assist in dispersing the powder pharmaceutical composition from the outlet for delivery to a subject through the outlet. In some instances, the powdered medicament may be stored within a storage compartment (of the inhaler), and wherein the powder pharmaceutical composition is transferred from the storage compartment, through the inlet and into the dispersion chamber. In certain cases, the inlet may be in fluid communication with an initial chamber, and wherein the powder pharmaceutical composition is received into the initial chamber prior to passing through the inlet and into the dispersion chamber.

In practice, a patient may prime an aerosolization device by puncturing a container holding the formulation (such as a capsule or blister), or the patient may transfer drug from a powder reservoir into the inhalation portion of the device, and then inhale. Inhalation by a patient draws the powder through the inhaler device where powder entrainment results in fluidization, and deagglomeration of powder agglomerates into respirable particles. This approach may be useful for effectively dispersing both binary and ternary carrier-based compositions, as well as formulations comprising engineered particles.

Exemplary devices for use in administering the dry powder composition include dry powder inhalers and metered dose inhalers such as, but not limited to TWISTHALER® (Merck), DISKUS® (GSK), HANDIHALER® (BI), AEROLIZER®, TURBUHALER® (AstraZeneca), FLEXHALER® (AstraZeneca), NEOHALER® (BREEZHALER®) (Novartis), PODHALER® (Novartis), EASYHALER® (Orion), NOVOLIZER® (Meda Pharma), ROTAHALER® (GSK), and others. As known to those skilled in the art, different devices will have different performance characteristics based on the device resistance, deaggregation mechanisms, adhesion of drug to the internal flow channels, and the ability of the patient to coordinate and inhale, among other factors.

In some embodiments, the dry powder compositions may be administered using a dry powder inhaler that comprises a dry powder deagglomerator, also referred to as a powder dispersion mechanism. Exemplary powder dispersion mechanisms are described in U.S. Patent Publication Nos. 2013/0340754 and 2013/0340747, which are incorporated herein by reference in their entirety. In some instances, such powder dispersion mechanisms may comprise a bead positioned within a chamber that is arranged and configured to induce a sudden, rapid, or otherwise abrupt expansion of a flow stream upon entering the chamber. In general, the chamber may be coupled to any form or type of dose containment system or source that supplies powdered active agent into the chamber.

In some instances, the powder dispersion mechanism may be coupled to a dry powder inhaler such as a commercially available device. The dispersion mechanism (dispersion chamber) may be adapted to receive an aerosolized powdered active agent from an inlet channel such as described, for example, in U.S. Patent Publication No. 2013/0340754, which is incorporated herein by reference in its entirety. The powder dispersion mechanism (dry powder deagglomerator) may be adapted to receive at least a portion of the aerosolized powdered active agent from the first chamber of the inhaler. The powder dispersion mechanism may include a dispersion chamber that may hold an actuator that is movable within the dispersion chamber along a longitudinal axis. The dry powder inhaler may include an outlet channel through which air and powdered active agent exit the inhaler to be delivered to a subject. A geometry of the inhaler may be such that a flow profile is generated within the dispersion chamber that causes the actuator to oscillate along the longitudinal axis, enabling the oscillating actuator to effectively disperse powdered medicament received in the dispersion chamber for delivery to the patient through the outlet channel.

In certain instances, a dry powder inhaler system may be used to aerosolize and administer the dry powder composition. The dry powder inhaler system may include a receptacle containing an amount of powdered active agent. The dry powder inhaler system may include an inlet channel that is adapted to receive air and powdered active agent from the receptacle. The dry powder inhaler system may include a first chamber that is adapted to receive air and powdered active agent from the inlet channel. A volume of the first chamber may be greater than volume of the inlet channel. The dry powder inhaler system may include a dispersion chamber that is adapted to receive air and powdered medicament from the first chamber. The dispersion chamber may hold an actuator that is movable within the dispersion chamber along a longitudinal axis. The dry powder inhaler system may include an outlet channel through which air and powdered active agent exit the dispersion chamber to be delivered to a patient. A geometry of the system may be such that a flow profile is generated within the system that causes the actuator to oscillate along the longitudinal axis, enabling the oscillating actuator to effectively disperse powdered medicament received in the dispersion chamber for delivery to the patient through the outlet channel.

### Administration and Dosage

Compositions and methods according to embodiments of the invention may provide for dose administration without a strict treatment regimen. Moreover, a medication designed to be used “as-needed” can provide a significant boost for patients who adhere poorly to their chronic medication regimen, or for those patients who have a deficit in exercise tolerance at various times in the day as a result of their treatment regimen, even if fully adherent. In this regard, a PRN therapeutic can decrease or otherwise improve symptoms for patients who miss a dose, while allowing them to maintain their scheduled treatment regimen. The use of a PRN therapeutic can simplify medication counseling on skipped doses, e.g., if a dose is missed, a patient can be counseled to administer a dose of the PRN therapeutic and then resume the normal treatment schedule for their chronic medication.

Upon administration of the PRN dosage form according to embodiments of the invention, a patient may begin to feel relief of their symptoms quickly—in some cases, relief is felt essentially immediately. PRN dosage forms according to embodiments of the invention may have a rapid onset of action, e.g., with a maximum concentration of drug attained in the circulation in less than 15 min, e.g., less than 10 min or less than 5 min. Moreover, the pharmacodynamic effects (e.g., improved hemodynamics, gas exchange, and symptom improvement) may occur, e.g., in under 30 min, with measurable improvements within 15 min or 10 min or less post-administration.

Administration of vasodilators via inhalation, as described herein, may provide a rapid onset of action with tₘₐₓ values for vasodilators well below one hour. For example, inhaled vardenafil has a tₘₐₓ of 2 min or less following pulmonary administration. In contrast, tₘₐₓ values for commonly oral vasodilators typically range from 2-8 hours. While intravenous injectables provide for immediate drug levels in the circulation, they are less suitable as PRN therapeutics, because dose delivery is highly invasive, provides high systemic drug levels leading to significant adverse events, and the treatment is inconvenient to administer by the patient on an as-needed basis without the use of complex pump systems.

Importantly, the PRN therapeutics according to embodiments of the invention may provide symptom relief and improvements in exercise tolerance over a duration long enough to complete a target activity. In general, the duration of action is at least 1 h, extending to more than 2 h, to more than 3 h, or to longer extended periods.

The PRN therapeutics according to embodiments of the invention may maximize the amount of drug delivered to the pulmonary arteries, while minimizing off-target delivery of the drug (e.g., gastrointestinal delivery and systemic delivery), to minimize safety and tolerability issues. Targeted delivery to the lungs is maximized by administration of aerosols via oral inhalation. A superior adverse event profile is a particular important advantage of a PRN therapeutic, given that the drug is administered on top of background therapies. Injectables lead to high systemic drug levels and to significant adverse events making them less suitable for PRN administration.

In methods according to embodiments of the invention, when a patient is anticipating physical exertion such as, for example, exercising, taking a walk, a trip to the store, or other activity, the subject may administer via inhalation the PRN formulation, comprising a vasodilator (e.g., a PDE5i drug) alone or combination with a second drug (either co-formulated or packaged separately). Typically, the patient may administer the formulation(s) 2-30 minutes before initiating such activity. The patient may administer these doses, e.g., in addition to any chronic therapies they are taking. The dose of the vasodilator, or the combination of drugs as described above, may be sufficiently low as to not cause significant additive side effects to the baseline drug regimen the patient is taking but nonetheless sufficient to transiently dilate pulmonary blood vessels for at least 30 minutes up to at least 6 hours, allowing patients to enhance their exercise tolerance and complete their desired ADLs.

In some embodiments, the PRN therapeutic comprises one or more active agents in a nominal dose ranging from 0.1 to 5.0 mg (e.g., from 0.15 to 0.5 mg). In some embodiments, the PRN therapeutic comprises one or more active agents in a nominal dose ranging from 0.02 to 1.0 mg (e.g., from 0.04 to 0.1 mg). As a non-limiting example, the nominal dose of vardenafil (in base form) for an inhaled PRN therapeutic formulated in a carrier-based formulation and delivered with a capsule-based dry powder inhaler may typically range from about 0.1 mg to about 2.0 mg, (e.g., between 0.15 and 0.50 mg).

Formulations of varied doses are contemplated, including, for example, formulations having inhaled dosages from 0.01 mg to 5 mg of active agent of PDE5i delivered to the lung.

For example, the PDE5i inhaled dose may be in the range of 0.01 mg to 0.5 mg, 0.01 mg to 1 mg, 0.01 mg to 2 mg, 0.025 mg to 0.5 mg, 0.025 mg to 1 mg, 0.025 mg to 2 mg, 0.05 mg to 0.5 mg, 0.05 mg to 1 mg, 0.05 mg to 2 mg, 0.075 mg to 0.5 mg, 0.075 mg to 1 mg, 0.075 mg to 2 mg, 0.1 mg to 0.25 mg, 0.1 mg to 0.5 mg, 0.1 mg to 1 mg, 0.1 mg to 2 mg, 0.1 mg to 3 mg, 0.1 mg to 4 mg, 0.25 mg to 0.5 mg, 0.25 mg to 0.75 mg, 0.25 mg to 1 mg, 0.25 mg to 1.5 mg, 0.25 mg to 2 mg, 0.25 mg to 3 mg, 0.25 mg to 4 mg, 0.5 mg to 0.75 mg, 0.5 mg to 1 mg, 0.5 mg to 2 mg, 0.5 mg to 1 mg, 0.5 mg to 2 mg, 0.5 mg to 3 mg, 0.5 mg to 4 mg, 0.75 mg to 1 mg, 0.75 mg to 2 mg, 0.75 mg to 1 mg, 0.75 mg to 2 mg, 0.75 mg to 3 mg, 0.75 mg to 4 mg, 1 mg to 1.5 mg, 1 mg to 2 mg, 1 mg to 2.5 mg, 1 mg to 3 mg, 1 mg to 3.5 mg, 1 mg to 4 mg, 2 mg to 3 mg, 2 mg to 4 mg, 0.5 mg to 4.5 mg, 2 mg to 5 mg, and doses within 25% of these ranges. In some such embodiments, the PDE5i drug is vardenafil.

In some instances, the PDE5i composition may have an inhaled dose of 0.01 mg, 0.25 mg, 0.05 mg, 0.075 mg, 0.1 mg, 0.125 mg, 0.15 mg, 0.175 mg, 0.2 mg, 0.225 mg, 0.25 mg, 0.275 mg, 0.3 mg., 0.325 mg, 0.35 mg, 0.375 mg, 0.4 mg, 0.425 mg, 0.45 mg, 0.5 mg, 0.525 mg, 0.55 mg, 0.575 mg, 0.6 mg, 0.625 mg, 0.65 mg, 0.675 mg, 0.7 mg, 0.725 mg, 0.75 mg, 0.775 mg, 0.8 mg, 0.825 mg, 0.85 mg, 0.875 mg, 0.9 mg, 0.925 mg, 0.95 mg, 0.975 mg, 1.0 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2.0 mg, 2.1 mg, 2.15 mg, 2.2 mg, 2.25 mg, 2.3 mg, 2.35 mg, 2.4 mg, 2.45 mg, 2.5 mg, 2.55 mg. 2.6 mg, 2.65 mg, 2.7 mg, 2.75 mg, 2.8 mg, 2.85 mg, 2.9 mg, 2.95 mg, 3.0 mg. 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4.0 mg, 4.1 mg, 4.15 mg, 4.2 mg, 4.25 mg, 4.3 mg, 4.35 mg, 4.4 mg, 4.45 mg, 4.5 mg, 4.55 mg, 4.6 mg, 4.65 mg, 4.7 mg, 4.75 mg, 4.8 mg, 4.85 mg, 4.9 mg, 4.95 mg, 5.0 mg, or a dose within 25% of any of these doses. In some such embodiments, the PDE5i drug is vardenafil.

In some instances, the PDE5i composition may have an inhaled dose of at least about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.4 mg, 3.5 mg, 3.6 mg. 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, or 5 mg. In some instances, the PDE5i composition may have an inhaled dose of no more than about 0.1 mg, no more than about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, or 5 mg. In some instances, the subject may receive a daily inhaled dose of the PDE5i composition of 0.25 mg to 1 mg, 0.1 mg to 4 mg, 0.1 mg to 2 mg, 1 mg to 4 mg, or a dose within 25% of these ranges. In some such embodiments, the PDE5i drug is vardenafil.

In some instances, the PDE5i composition may have an inhaled dose of at least about 0.001 mg, 0.0025 mg, 0.005 mg, 0.0075 mg, 0.01 mg, 0.0125 mg, 0.015 mg, 0.0175 mg, 0.02 mg, 0.025 mg, 0.0275 mg, 0.03 mg, 0.0325 mg, 0.035 mg, 0.0375 mg, 0.04 mg, 0.0425 mg, 0.05 mg, 0.0525 mg, 0.055 mg, 0.0575 mg, 0.06 mg, 0.0625 mg, 0.065 mg, 0.0675 mg, 0.07 mg, 0.0725 mg, 0.075 mg, 0.0775 mg, 0.08 mg, 0.0825 mg, 0.085 mg, 0.0875 mg, 0.09 mg, 0.0925 mg, 0.095 mg, 0.0975 mg, 0.1 mg, 0.125 mg, 0.15 mg, 0.175 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5, or a dose within 25% of any of these doses. In some such embodiments, the PDE5i drug is vardenafil.

In some instances, the PDE5i composition may have an inhaled dose of no more than about 0.001 mg, 0.0025 mg, 0.005 mg, 0.0075 mg, 0.01 mg, 0.0125 mg, 0.015 mg, 0.0175 mg, 0.02 mg, 0.025 mg, 0.0275 mg, 0.03 mg, 0.0325 mg, 0.035 mg, 0.0375 mg, 0.04 mg, 0.0425 mg, 0.05 mg, 0.0525 mg, 0.055 mg, 0.0575 mg, 0.06 mg, 0.0625 mg, 0.065 mg, 0.0675 mg, 0.07 mg, 0.0725 mg, 0.075 mg, 0.0775 mg, 0.08 mg, 0.0825 mg, 0.085 mg, 0.0875 mg, 0.09 mg, 0.0925 mg, 0.095 mg, 0.0975 mg, 0.1 mg, 0.125 mg, 0.15 mg, 0.175 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5, or a dose within 25% of any of these doses. In some instances, the subject may receive a daily inhaled dose of 0.003 mg to 1 mg, 0.015 mg to 0.75 mg, 0.075 mg to 0.375 mg, 0.075 mg to 0.75 mg, or a dose within 25% of these ranges. In some such embodiments, the PDE5i drug is vardenafil.

The term “nominal dose” or “total dose” refers to the total amount or mass of active agent packaged or partitioned for administration to a subject. For example, the nominal dose is the total amount of active agent that is enclosed in a capsule for use with an inhaler.

### Example 1

RT234 designates a drug/device combination (Respira Therapeutics, Palo Alto, CA, USA) that delivers the phosphodiesterase 5 inhibitor vardenafil to the lungs via inhalation and has been shown to reduce pulmonary vascular resistance (PVR) in patients with PAH. This example describes a study that aims to evaluate whether RT234 can increase oxygen capacity during cardiopulmonary exercise testing (CPET) in patients with PAH.

This prospective, multi-center, open-label, two-cohort, dose-escalation, phase IIb trial in patients with PAH will evaluate the safety and efficacy of RT234 in improving exercise parameters. Patients eligible for enrollment will have a right heart catheterization (RHC)-confirmed diagnosis of PAH, a 6-minute walking distance of ≥150 m, a minute ventilation/carbon dioxide production (VE/VCO₂) slope of ≥36, and will be on up to three stable oral and/or inhaled (not parenteral) PAH-specific background therapies. The estimated sample size is 86 patients, who will be divided into two dose cohorts. Cohort 1 will receive 0.5 mg RT234, and cohort 2 will receive 1.0 mg RT234. Each cohort will contain two subgroups based on the number of PAH background medications (up to two vs three). The trial will assess patients' changes from baseline in peak oxygen consumption (VO₂) during CPET 30 minutes after a single dose of 0.5 mg or 1.0 mg RT234, the change in the 6-minute walking distance, and the pharmacokinetics and safety profile of single doses of RT234.

To be eligible for inclusion in the trial, patients must be between 18 and 80 years of age, inclusive: have a diagnosis of right heart catheterization (RHC)-confirmed PAH in any of the following: (1) idiopathic, primary, or familial PAH, or (2) PAH associated with connective tissue diseases, or (3) PAH associated with: HIV; simple, congenital systemic-to-pulmonary shunts ≥1-year post-surgical repair; exposure to drugs, chemicals, and toxins; the patient must have had ventilation/perfusion scan, computerized tomography angiogram, or pulmonary arteriogram that rules out chronic thromboembolic pulmonary hypertension; previous PAH diagnosis with the following conditions: (1) stable PAH without significant adjustments of disease-specific background PAH therapy ≥3 months before the CPET procedure and (2) if on corticosteroids, has been receiving a stable dose of ≤20 mg per day of prednisone (or equivalent dose of other corticosteroid) for ≥30 days before the baseline CPET; pulmonary function testing (PFT) within 6 months before commencement of screening, or during the screening period, that fulfills the following: (1) forced expiratory volume in 1 second (FEV₁)≥60% predicted, (2) forced expiratory vital capacity (FVC)≥60% predicted, (3) FEV₁/FVC≥60%; RHC performed and documented before screening that is consistent with the diagnosis of PAH, meeting all the following criteria: (1) mPAP≥20 mmHg (at rest), and (2) PCWP or left ventricular end-diastolic pressure of ≤12 mmHg if PVR is ≥300 to <500 dyn·s cm⁻⁵, or PCWP or LVEDP≤15 mmHg if PVR is >500 dyn·s cm⁻⁵ and, if PCWP is not available, then mean left arterial pressure (mLAP) or LVEDP≤15 mmHg or ≤12 mmHg in the absence of left atrial obstruction, and PVR>3 Wood units or >240dyn·s cm⁻⁵; WHO/New York Heart Association functional class II-IV symptomatology; have a body mass index≤35.9 kg m−²; be on stable PAH disease-specific background therapy of up to three oral and/or inhaled therapies; have a 6MWD of ≥150 m; and have minute ventilation (VE)/carbon dioxide production (VCO₂) slope of ≥36 during the baseline CPET; maximal effort on the baseline CPET must reach a peak respiratory exchange ratio (RER) of ≥1.0. If the patient is taking the following concomitant medications, which may affect PAH, the patient must be on a stable therapeutic dose for ≥1 month before the screening, and the dosage must be maintained throughout: (1) vasodilators, (2) digoxin; (3) L-arginine supplement; (4) anticoagulants (anticoagulation status should be maintained/stable in the therapeutic range for ≥1 month before the screening). Stable may be defined as no change in PAH-specific drug therapy within 3 months of screening visit 1 and for the duration of the study, and no change in dose of PAH-specific drug(s) within 1 month of screening. Individuals with a body mass index ≥36.0 kg m−² may be considered for inclusion in the trial.

Patients will be excluded from the trial if they have baseline systemic hypotension, defined as mean arterial pressure <50 mmHg or systolic blood pressure (SBP)<90 mmHg at screening; a history of or current uncontrolled hypertension, defined as SBP>175 mmHg or sitting diastolic blood pressure>110 mmHg; a history of chronic uncontrolled asthma; patients who are unable or may find it difficult to use an inhaler device; requirement for intravenous inotropes within 30 days before the baseline CPET procedure; use parenteral PAH medications; use of ricioguat as background PAH therapy ≤1 month before initiating screening or during the study through the end of visit 4; use of oral, topical, or inhaled nitrates within 2 weeks before the baseline CPET procedure; portopulmonary hypertension, portal hypertension, or chronic liver disease determined to be Child-Pugh B or C; history of atrial septostomy; history of known uncorrected right-to-left shunt; clinically relevant, persistent patent foramen ovale; or known Eisenmenger's physiology; paroxysmal or uncontrolled atrial fibrillation; chronic renal insufficiency; serum alanine aminotransferase or aspartate aminotransferase that is ≥3× the upper limit of the normal range; platelets<50,000 μL⁻¹ at screening; hemoglobin concentration<9 gdL⁻¹ at screening; or have evidence or history of left-sided heart disease and/or clinically significant cardiac disease.

Once patient eligibility has been confirmed, the study will begin with the screening visit(s) (visit 1), which will occur between 28 and 3 days before the CPET baseline visit (FIG. 2). The screening visit(s) will include two 6-minute walking tests (6MWTs) performed at least 2 days apart or, if tolerable to the patient, at least 2 hours apart on the same day (this will account for the learning effect associated with the 6MWT: the relative difference between the two measurements must be ≤15%). The two screening 6MWDs will be averaged to determine the baseline 6MWD. If the relative difference between the two measurements is >15%, a third 6MWT may be performed. If a third 6MWT is performed, then the two longest 6MWTs will be averaged to determine the baseline 6MWD. Urine or serum pregnancy test may also be conducted at screening visit 1. Patient height may also be obtained at screening visit 1.

At the CPET baseline visit (visit 2; day 1), enrolled patients will return to the testing center for pre-test assessments, baseline CPET to measure peak oxygen consumption (VO₂), and post-test assessments. Eligible patients whose baseline CPETs are deemed evaluable by the CPET Core Laboratory will enter the treatment period (visits 3 and 4) for dosing with RT234 and post-dose CPET and 6MWT. Prothrombin time international normalized ratio may also be tested in patients taking oral vitamin K antagonists.

At the CPET treatment visit (visit 3; day 8), patients will return to the testing facility for pre-CPET assessments and a single dose of RT234 (0.5 mg for cohort 1 and 1.0 mg for cohort 2), which will be administered with the aid of the study team. CPET will be performed 30 minutes post dose, at approximately the same time of day at each visit (within 2 hours), followed by post-test assessments. The primary endpoint may be the change in peak VO₂ from day 1 to day 8 (30 minutes post dose). Urine or serum pregnancy test may also be conducted at visit 3.

At the 6MWT treatment visit (visit 4; day 15), patients will undergo pre-6MWT assessments and receive a single dose of RT234. Patients will perform a 6MWT 30 minutes post dose and will then undergo post-test assessments. Plasma samples for PK analyses will be collected before and after RT234 dosing at visits 3 and 4. Urine or serum pregnancy test may also be conducted at visit 4.

RT234 will only be administered to patients during visits 3 and 4 (FIG. 1); patients will not use RT234 outside these two visits. Patients will remain at the clinic for PK sampling and safety monitoring for 4 hours once all tests have been completed during visits 3 and 4, and safety monitoring will continue for 30 days after visit 4. Visit 5 (day 45±3-day window) will involve a follow-up telephone call assessment of safety.

For statistical analysis, the estimated total sample size is 86 enrolled patients, with a 5% dropout rate and a computed sample size of 17 and 26 patients with up to two and three background medications, respectively, per dose cohort (0.5 and 1.0 mg). This was derived using a one-sample two-sided t test (0.05 significance level and 80% power) to test the null hypothesis of no change in peak VO₂ from baseline to post dose assessed during CPET performed 30 minutes after RT234 dosing. The assumed mean change from baseline is 1.5 and 1.2 mL O₂kg⁻¹·min⁻¹ for patients with up to two and three background medications, respectively, in each dose cohort. A standard deviation of 2 mL O₂·kg⁻¹ ·min⁻¹ is assumed for each dose cohort and background medication group.

The primary efficacy analysis will be conducted on the modified intention-to-treat (mITT) analysis set, which comprises all treated patients with baseline and post-baseline assessment of peak VO₂. This mITT analysis set will serve as the basis for all efficacy analyses. The analysis procedure will be repeated for the per-protocol analysis set (all mITT patients who do not experience any major protocol violations) using a one-sample t test (if peak VO₂ follows a normal distribution) to test the two-sided null hypothesis (0.05 significance level) of no change in peak VO₂. The 95% CI for peak VO2 mean change will be derived from the t test if the normality assumption is not rejected. If it is rejected, a one-sample Wilcoxon signed-rank test will be used to determine the median change in peak VO2 with a 95% CI estimate.

The secondary efficacy analyses will be conducted on the mITT analysis set and will be repeated with the per-protocol analysis set, as described for the primary efficacy analyses.

The PK parameters of vardenafil and the relationship between vardenafil exposure and changes in CPET parameters and 6MWD will also be determined. Plasma samples will be collected at visits 3 and 4 before RT234 dosing; at 3, 15, and 30 minutes post dose; immediately at the end of the exercise period; and at 45, 75, 120, 180, and 240 minutes post dose. Vardenafil concentrations will be measured in the PK samples as described previously. Estimators of PK parameters will include Tₘₐₓ, Cₘₐₓ, area under the curve from time 0 to the last measurable concentration (AUC_{0-Last}), AUC from time 0 to infinity (AUC_{0-Inf}), and half-life (t_{1/2}). For CPET, exposure-response analysis will be based on AUC0_{-Last} and change from baseline in peak VO₂ measured during CPET 30 minutes after RT234 dosing; for the 6MWT, exposure-response analysis will be based on AUC_{0-Last} and change from baseline 6MWD (mean of the screening 6MWDs) to the 6MWD 30 minutes after RT234 dosing.

Planned PK parameters (Tₘₐₓ, Cₘₐₓ, AUC_{0-Last}, AUC_{0-Inf}, and t_{1/2}) will be calculated for treated patients with available PK measurements using the standard linear trapezoidal convention for all available drug concentration measurements. Standard descriptive statistics (mean, median, n, standard deviation, minimum, maximum, and coefficient of variation) will be used to summarize the PK parameters, along with the 90% CI, when appropriate.

The primary efficacy endpoint is the change from baseline in peak oxygen consumption (VO₂) measured during CPET performed 30 minutes after a single dose of 0.5 mg or 1.0 mg RT234. The secondary efficacy endpoints are change from baseline in 6MWD (mean of two 6MWDs at screening) to the 6MWD measured 30 minutes after RT234 dosing; change from baseline to post dose in the minute VE/VCO₂ slope during CPET; change from baseline to post dose in the response of the partial pressure of end-tidal carbon dioxide apex to exercise (i.e., highest level during CPET); change from baseline to post dose in the duration of exercise during CPET; change from baseline to post dose in peak perceived dyspnea during CPET, as assessed by the Modified Borg Dyspnea Scale Score; change from baseline to post dose in Patient Global Impression of Severity (PGI-S) for CPET (assessed before CPET and after a 2-minute cooldown after treadmill exercise while wearing mask, and then at 5-minute intervals [7, 12, and 17 minutes] without a mask); and changes from screening to post dose in PGI-S for 6MWT (assessed before the 6MWT and at 2 minutes after completion of the 6MWT).

Exploratory endpoints may, for example, include the proportion of patients with improvement in risk category (low, intermediate, or high) for the VE/VCO₂ slope criteria during CPET from baseline to post dose, and changes in the following additional CPET parameters from baseline to post dose: electrocardiogram (ECG) response to exercise, VO₂ at ventilatory threshold, change in respiratory exchange ratio at peak VO₂, SBP response to exercise, pulse oximetry response to exercise, the non-peak Modified Borg Dyspnea Scale Score throughout exercise (at every non-peak minute during CPET and for 6 minutes post CPET), the Borg Rating of Perceived Exertion Scale throughout exercise (every 2 minutes during CPET and for 6 minutes post CPET), the Duke Activity Status Index (may be measured for 10 minutes post CPET), and the 4-Point Angina Scale (every 2 minutes during CPET and for either 6 minutes post CPET or, if indicated, until symptom recovery).

The assessment of safety will involve evaluating the adverse event profile and acute physical and cardiac symptoms of single doses (0.5 or 1.0 mg) of RT234. The incidence and severity of TEAEs will be summarized by Medical Dictionary for Regulatory Activities (current version) System Organ Class and Preferred Term, National Cancer Institute Common Terminology Criteria for Adverse Events (current version) grade, and causality (attribution to study treatment and related/not related to investigational medicinal product). Changes in vital signs (blood pressure [BP], heart rate, respiratory rate, body temperature, and pulse oximetry) will be monitored, and physical examinations and 12-lead electrocardiograms (ECGs) will be conducted to measure the change from baseline to post dose. Interval history will include any signs, symptoms, or events experienced by the patient since the previous study visit. Vital signs may be tested after resting for 5 minutes (sitting). Vital signs should be taken before any blood draw. Vital signs may also be measured during CPET and into recovery. Hear rate may be measured via continuous ECG monitoring during CPET and for 6 minutes post CPET. BP assessment may be measured every 2 minutes during CPET and for 6 minutes post CPET. Pulse oximetry may be measured every minute during CPET and for 6 minutes post CPET. Vital signs may also be measured at treatment 6MWT visit 4 (before RT234 dosing; 5 and 15 minutes post dose before 6MWT at 30 minutes post dose; and at 60 and 120 minutes post dose).

### Example 2

This example describes a study that aims to evaluate whether RT234 can increase oxygen capacity during cardiopulmonary exercise testing (CPET) in patients with PAH. The study in this Example differs from the study in Example 1 only in that the trial will involve an estimated sample size of 37 patients, who will be divided into 3 dose cohorts. Cohort 1 will consist of an estimated 7 patients who will receive 0.5 mg RT234; cohort 2 will consist of an estimated 15 patients who will receive 1.0 mg RT234; and cohort 3 will consist of an estimated 15 pts who will receive 2.0 mg RT234. Also, this study will exclude patients who have a baseline peak VO₂ of >15 mL/mg/kg. In all other significant respects, the study of this Example will be similar to the study of Example 2.

1. A method of treating pulmonary hypertension, the method comprising administering to a subject in need thereof an effective amount of vardenafil or a pharmaceutically acceptable salt or hydrate thereof,
wherein the vardenafil is administered via inhalation pro re nata using a portable inhaler, and
wherein the vardenafil is administered at least 2-30 minutes before physical exertion.

2. The method of claim 1, wherein administering the vardenafil comprises targeting the vardenafil to the small airways of the lungs in the subject.

3. The method of claim 2, wherein a nominal dose of about 0.5 mg to about 1.0 mg of vardenafil is delivered to the subject upon inhalation.

4. The method of claim 3, wherein a nominal dose of about 0.5 mg of vardenafil is delivered to the subject upon inhalation.

5. The method of claim 3, wherein a nominal dose of about 1.0 mg of vardenafil is delivered to the subject upon inhalation.

6. The method of claim 2, wherein a nominal dose of about 0.5 mg to about 2.0 mg of vardenafil is delivered to the subject upon inhalation.

7. The method of claim 6, wherein a nominal dose of about 2.0 mg of vardenafil is delivered to the subject upon inhalation.
